(19) United States
(12) Patent Application Publication
(10) Pub. No.: US 2024/0100287 A1
(43) Pub. Date: Mar. 28, 2024

(54) HYBRID SINGLE-LIMB MEDICAL VENTILATION

(71) Applicant: Covidien LP, Mansfield, MA (US)

(72) Inventors: Gabriel Sanchez, Valley Center, CA (US); Richard J. Nakai, Long Beach, CA (US); Nancy F. Dong, San Marcos, CA (US); Kun Li, San Diego, CA (US)

(73) Assignee: Covidien LP, Mansfield, MA (US)

(21) Appl. No.: 18/466,338

(22) Filed: Sep. 13, 2023

Related U.S. Application Data
(60) Provisional application No. 63/410,027, filed on Sep. 26, 2022.

(51) Int. Cl. A61M 16/08; A61M 16/00; A61M 16/16; A61M 16/20

(52) U.S. Cl. CPC A61M 16/0883; A61M 16/0003; A61M 16/026; A61M 16/16; A61M 16/208; A61M 2016/0027; A61M 2016/0042; A61M 2205/15; A61M 2230/40; A61M 2230/43

(57) ABSTRACT

A hybrid single-limb patient circuit coupled to an inspiratory port and an expiratory port of a ventilator. The hybrid single-limb patient circuit may include a check valve positioned to direct breathing gases supplied from the inspiratory port in a single direction; a manifold pneumatically coupled to the check valve; a dual-purpose single limb, pneumatically coupled to the manifold and the non-invasive patient interface, to carry breathing gases to the non-invasive patient interface and carry exhaled gases from the non-invasive patient interface; and an exhalation tubing segment, pneumatically coupled to the manifold and the expiratory port, to carry the exhaled gases from the manifold to the expiratory port.

### CROSS-REFERENCE TO RELATED APPLICATIONS

This application claims the benefit of U.S. Provisional Application No. 63/410,027 filed Sep. 26, 2022, entitled “Hybrid Single-Limb Medical Ventilation,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. Different types of tubing arrangements exist for providing the breathing gases to the patient.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to a ventilation system that includes a ventilator, which includes an inspiratory port, an expiratory port, and an exhalation valve positioned at the expiratory port. The ventilation system also includes a non-invasive patient interface and a hybrid single-limb patient circuit coupled to the inspiratory port and the expiratory port. The hybrid single-limb patient circuit includes a check valve positioned to direct breathing gases supplied from the inspiratory port in a single direction; a manifold pneumatically coupled to the check valve; a dual-purpose single limb, pneumatically coupled to the manifold and the non-invasive patient interface, to carry breathing gases to the non-invasive patient interface and carry exhaled gases from the non-invasive patient interface; and an exhalation tubing segment, pneumatically coupled to the manifold and the expiratory port, to carry the exhaled gases from the manifold to the expiratory port.

In an example, the manifold includes a post-valve inhalation segment to carry the breathing gases passing through the check valve; a post-manifold single-limb segment to carry the breathing gases from the post-valve inhalation segment to a single-limb port and carry the exhaled gases received from the single-limb port; and an adapter exhalation segment to carry the exhaled gases to the exhalation tubing segment. In another example, the ventilation system includes a humidifier, and the check valve is coupled to an output of the humidifier. In yet another example, the ventilator further includes an expiratory flow sensor; an expiratory pressure sensor; an inspiratory pressure sensor; an inspiratory flow sensor; a processor; and memory, the memory storing instructions that, when executed by the processor, cause the ventilator to perform operations. The operations include: based on measurements from the expiratory flow sensor, estimate an exhaled volume of gas; estimate a volume of supplied breathing gas; based on the estimated exhaled volume of gas, the estimated volume of supplied breathing gas, and at least one of an inspiratory pressure measurement from the inspiratory pressure sensor or an expiratory pressure measurement from the expiratory sensor, estimating a leak rate. In a further example, the operations also include maintain, during an exhalation phase, a positive end expiratory pressure (PEEP) level by: controlling an exhalation valve; and supplying breathing gases through the inspiratory port at a flow rate based on the estimated leak rate. In still another example, the operations further include: estimate a compliance of the hybrid single-limb patient circuit; based on the compliance, an expiratory flow measurement from the expiratory sensor, the exhaled volume of gas, and, the leak rate, estimating a volume of rebreathed carbon dioxide.

In another aspect, the technology relates to a hybrid adapter for facilitating hybrid single-limb ventilation. The hybrid adapter includes a breathing gas input port to receive breathing gases from a ventilator; a pre-valve inhalation segment coupled to the breathing gas input port; a check valve, coupled to the pre-valve inhalation segment, to direct the breathing gases in a single direction; a manifold; a single-limb port, coupled to the manifold, to deliver the breathing gases to a couplable dual-purpose single limb and receive exhaled breathing gases from the couplable dual-purpose single limb; and an output port, coupled to the manifold, to deliver the exhaled gases to a couplable exhalation tubing segment.

In an example, the manifold includes a post-valve inhalation segment coupled to the check valve; a post-manifold single-limb segment extending from the post-valve inhalation segment to the single-limb port; and an adapter exhalation segment extending from the post-manifold single-limb segment to the output port. In another example, the pre-valve inhalation segment, the post-valve inhalation segment, the post-manifold single-limb segment, and the adapter exhalation segment comprise tubing. In still another example, the pre-valve inhalation segment, the post-valve inhalation segment, the post-manifold single-limb segment, and the adapter exhalation segment comprise bores in a housing of the hybrid adapter.

In another aspect, the technology relates to a ventilator-implemented method including estimating a volume of supplied breathing gas flowing from an inspiratory port of a ventilator and into a hybrid single-limb patient circuit, the hybrid single-limb patient circuit comprising a dual-purpose single limb defining a lumen that carries both delivered breathing gases and returned exhaled gases; estimating a volume of exhaled gas flowing from the hybrid single-limb patient circuit into an expiratory port of the ventilator; estimating a circuit pressure of the hybrid single-limb patient circuit at a patient interface; based on the estimated exhaled volume of gas, the estimated volume of supplied breathing gas, and the circuit pressure, estimating a leak rate; based on the estimated leak rate, maintaining a positive end expiratory pressure (PEEP) level in the hybrid single-limb patient circuit during an exhalation phase.

In an example, estimating the volume of exhaled gases is based on measurements from an expiratory flow sensors positioned at the expiratory port. In another example, maintaining PEEP includes controlling a closure rate of an exhalation valve at the expiratory port; and supplying breathing gases through the inspiratory port at a flow rate based on the estimated leak rate. In yet another example, the method includes estimating an amount of rebreathed carbon dioxide based on the leak rate and at least one of volume of supplied breathing gas or the volume of exhaled gases. In still another example, the method further includes determining a compliance of the hybrid single-limb patient circuit, and wherein estimating the amount of rebreathed carbon dioxide is further based on the determined compliance.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts a diagram illustrating an example of a medical ventilation system connected to a human patient using a hybrid single-limb patient circuit.

FIG. 2 depicts a schematic diagram of a medical ventilation system with a hybrid single-limb patient circuit.

FIG. 3 depicts a schematic diagram of the medical ventilation system of FIG. 2 during an inhalation phase of a breath.

FIG. 4 depicts a schematic diagram of the medical ventilation system of FIG. 2 during an exhalation phase of a breath.

FIG. 5 depicts a schematic diagram of the medical ventilation system of FIG. 2 during an exhalation phase of a breath where positive end expiratory pressure (PEEP) is maintained.

FIG. 6 depicts a perspective view of a medical ventilation system connected to a human patient using a hybrid single-limb patient circuit.

FIG. 7 depicts an example method for delivering ventilation with a hybrid single-limb patient circuit.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates.

Connection of the ventilator to the patient is achieved through tubing between the patient and the ventilator, which may be referred to as a patient circuit. In some examples, a single-limb patient circuit is implemented where only a single tube or limb extends from the ventilator to the patient. For instance, single-limb ventilation is sometimes performed by a ventilator that includes only an inspiratory port (without an exhalation port). As an example, single-limb ventilation may be performed by a ventilator having a blower. When the blower is activated during inhalation, air is delivered through the single limb to the patient. When the blower is deactivated (or slowed) during exhalation, the exhaled air from the patient is vented from the patient interface (e.g., a mask), and some of the exhaled gas travels back through the single limb back towards the inspiratory port of the ventilator. The exhaled gases may even travel back through the blower or other elements of the ventilator, which may lead to contamination.

To minimize the amount of return gas through the inspiratory port, a higher flow rate may be maintained during the exhalation phase of a breath (e.g., the blower remains at higher speed). While increasing the flow rate reduces the amount of return gas, the higher flow rate also increases the work of breathing (WOB) for the patient by providing additional resistance to the exhaled gases. Tradeoffs must then be made based on the WOB, receiving contaminated exhaled gases, and the potential for the patient rebreathing exhaled gases. In addition, with a traditional single-limb ventilation, pressures generally cannot be well maintained during the exhalation phase and the number of ventilation modes are substantially limited due to no use of exhalation limb or expiratory port.

Other examples of patient circuits use a dual-limb circuit, where an inhalation limb and an exhalation limb are implemented. The inhalation limb extends from the inspiratory port of the ventilator to a wye piece or patient interface, and the exhalation limb extends from the wye piece or patient interface to an expiratory port of the ventilator. The inhalation limb is used to deliver fresh breathing gas to the patient, and the exhalation limb is used to return exhaled gas from the patient. The dual-limb circuit helps is ensure a constant supply of fresh gas to the patient without the potential of rebreathing exhaled gas.

Unlike a single-limb circuit, the dual-limb circuit uses an exhalation valve to control circuit pressure during inhalation and exhalation phases of a breath. The exhalation system (e.g., exhalation limb, expiratory port, etc.) may also include sensors that measure gas properties, such as pressure and flow. Accordingly, by using a dual limb circuit, the flow of exhaled gas can be measured allowing the estimation of exhaled gas volume (exhaled tidal volume, VTE). The dual-limb circuit also provides the ability to estimate the amount of gas that was leaked during delivery, which allows for the potential to correct for estimation of delivered volume to the patient. Delivered and monitored data may then be compensated for the estimated leak. The use of a dual-limb circuit also provides for the ability to monitor pressure at both ends of the circuit to allow for a more accurate estimation of pressure at the wye piece or patient interface of the circuit.

Both single-limb circuits and dual-limb circuits have advantages and drawbacks. For instance, the dual-limb circuit allows for more measurements and data to be acquired and can facilitate additional modes of operation for the ventilator. The dual-limb circuit, however, adds weight, size, complexity, and cost to ventilation system. The single-limb circuit provides the advantage of simplicity and reducing the weight, size, and cost of the ventilation system. Yet, the single-limb circuit loses the advantages of the dual-limb circuit and also introduces additional drawbacks, such as potential contamination, rebreathing of gas, and/or increased work of breathing.

The present technology introduces a hybrid single-limb patient circuit that captures advantages of both single-limb ventilation and dual-limb ventilation. The hybrid single-limb patient circuit includes a check valve and a pneumatic manifold that allows for breathing gases to be delivered to the patient and received from the patient using a single limb, but still providing the benefits of the dual-limb circuits. For example, an inhalation tubing segment from an inspiratory port of ventilator may connect to a hybrid adapter. The hybrid adapter includes a pre-valve inhalation segment, a check valve, and a post-valve inhalation segment. Gases supplied to the hybrid adapter from the inhalation segment flow through the pre-valve inhalation segment, the check valve, and the post-valve inhalation segment. The post-valve inhalation segment connects to a manifold that branches to a post-manifold single-limb segment and an adapter exhalation segment. Supplied breathing gases flow out of the hybrid adapter via the post-manifold single-limb segment and into a dual-purpose single limb that couples to a patient interface. Exhaled gases are carried by the same dual-purpose single limb back into the hybrid adapter, where the manifold diverts the exhaled gases into an exhalation tubing segment coupled to the hybrid adapter. The exhalation tubing segment carries the exhaled gases into an expiratory port where properties of the exhaled gases may be measured.

Accordingly, from the perspective of the ventilator, the hybrid single-limb patient circuit appears similar to a dual-limb circuit, but from the perspective of the patient, the hybrid single-limb patient circuit appears as a single limb circuit. Thus, the benefits of a light-weight, low-cost single-limb circuit can be retained while also achieving the operational benefits of the dual-limb circuit. For instance, ventilation modes that operate based on the use of an exhalation valve or determined gas properties of exhaled gases may be used with the hybrid single-limb patient circuit of the present technology.

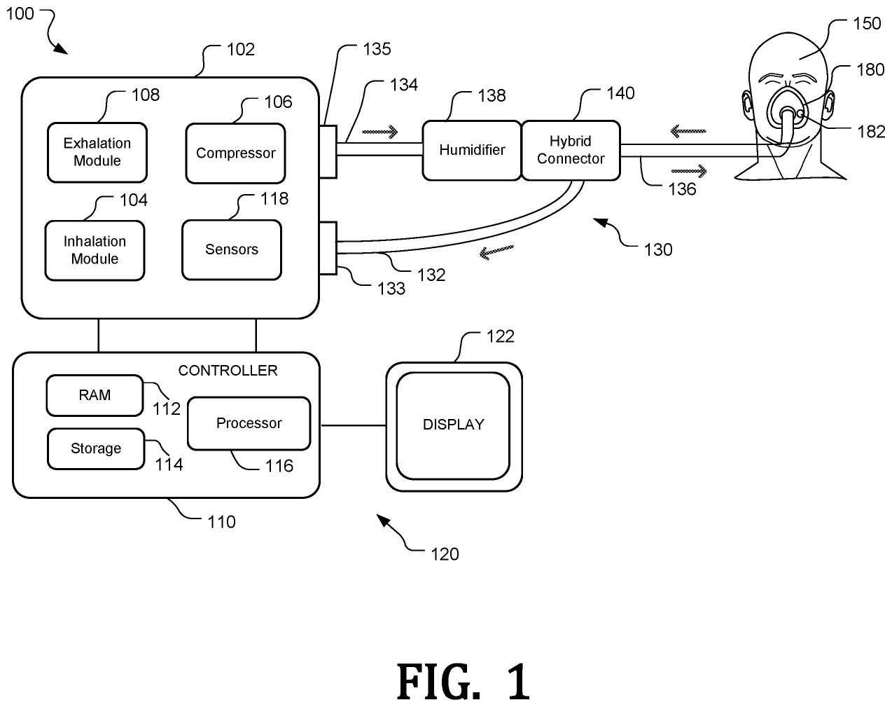

FIG. 1 is a diagram illustrating an example of a medical ventilation system 100 connected to a human patient 150. The ventilation system 100 may provide positive pressure ventilation to the patient 150. Ventilation system 100 includes a pneumatic system 102 (also referred to as a pressure generating system 102) for circulating breathing gases to and from patient 150 via the hybrid ventilation tubing system 130, which couples the patient to the pneumatic system via a non-invasive (e.g., mask, shown) patient interface 180 or an invasive (e.g., endotracheal tube) patient interface 180. The hybrid ventilation tubing system 130 may also be referred to as a hybrid single-limb patient circuit 130. Where ventilation is delivered using the hybrid single-limb patient circuit 130 and a non-invasive patient interface 180, the ventilation may be referred as hybrid single-limb non-invasive ventilation (NIV). The ventilation system 100 may also include a controller 110 and an operator interface 120 that includes a display 122. The pneumatic system 102, controller 110, and/or operator interface 120 may collectively be referred to as a ventilator.

The hybrid single-limb patient circuit 130 includes an inhalation tubing segment 134 that is coupled to an inspiratory port 135 of the pneumatic system 102. The hybrid single-limb patient circuit 130 also includes an inhalation tubing segment 134 that is coupled to an inspiratory port 135 of the pneumatic system 102. The inhalation tubing segment 134 carries breathing gases from the inspiratory port 135 to a humidifier 138 and/or a hybrid adapter 140.

The humidifier 138 humidifies the breathing gases before the gases reach the human patient 150. In the example depicted, the hybrid adapter 140 is positioned at an outlet of the humidifier 138. In other examples, however, the hybrid adapter 140 may be positioned at different locations within the hybrid single-limb patient circuit 130. As discussed further below, positioning the hybrid adapter 140 after the humidifier 138 (e.g., between the humidifier 138 and the patient interface 180), exhaled gases may be prevented from flowing back through the humidifier 138, which may cause contamination of the humidifier 138.

The hybrid adapter 140 pneumatically couples the inhalation tubing segment 134, the exhalation tubing segment 132, and a dual-purpose single limb 136 of the hybrid single-limb patient circuit 130. The dual-purpose single limb 136 carries fresh breathing gases to the patient 150 during inhalation phases and carries exhaled gases from the patient 150 during exhalation phases. For instance, during an inhalation phase, breathing gases are delivered through the inhalation tubing segment 134, the humidifier 138, the hybrid adapter 140, and the dual-purpose single limb 136 to the patient interface 180, where the patient 150 breathes the breathing gases. During an exhalation phase (e.g., when the patient 150 exhales), a portion of the exhaled gases are vented out to the room via a vent port 182 in the patient interface 180. The remainder of the exhaled gases are carried by the dual-purpose single limb 136 into the hybrid adapter 140, through the exhalation tubing segment 132, and then through the expiratory port 133. In an example, the dual-purpose single limb 136 defines a single lumen that carries both fresh breathing gases and exhaled gases.

As discussed further below, the hybrid adapter 140 includes a check valve that allows breathing gases to flow through the hybrid adapter 140 in a direction towards the patient 150 but prevents exhaled gases from flowing back through the hybrid adapter 140 towards the inspiratory port 135. Accordingly, exhaled gases are prevented from contaminating elements that located behind (e.g., closer to the ventilator) the hybrid adapter 140. In the example depicted, the ventilator and the humidifier 138 are thus protected, by the hybrid adapter 140, from the exhaled gases.

The pneumatic system 102 may have a variety of configurations. In the present example, pneumatic system 102 includes an exhalation module 108 coupled to the exhalation tubing segment 132 and an inhalation module 104 coupled with the inhalation tubing segment 134. Compressor 106 or other source(s) of pressurized gases (e.g., air, oxygen, and/or helium) is coupled with inhalation module 104 to provide a gas source for ventilatory support. The pneumatic system 102 may include a variety of other components, including mixing modules, valves, sensors, tubing, accumulators, filters, etc. For instance, the pneumatic system 102 may include gas property sensors 118, which may be internal or external sensors to the ventilator (and may be communicatively coupled, or capable communicating, with the ventilator). The gas property sensors 118 may include at least one sensor at or in the expiratory port 133 to measure properties of the exhaled gases flowing into the expiratory port 133. For instance, the expiratory port 133 may include a flow sensor and/or a pressure sensor. The expiratory port 133 also includes an expiratory valve that may be proportionally opened or closed to adjust the pressure and/or flow of the gases within the hybrid single-limb patient circuit 130.

The controller 110 is operatively coupled with pneumatic system 102, signal measurement and acquisition systems, and the operator interface 120 that may enable an operator to interact with the controller 110 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilation system 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. The memory 112 may store instructions that, when executed by the processor 116, cause the ventilator to perform the operations discussed herein.

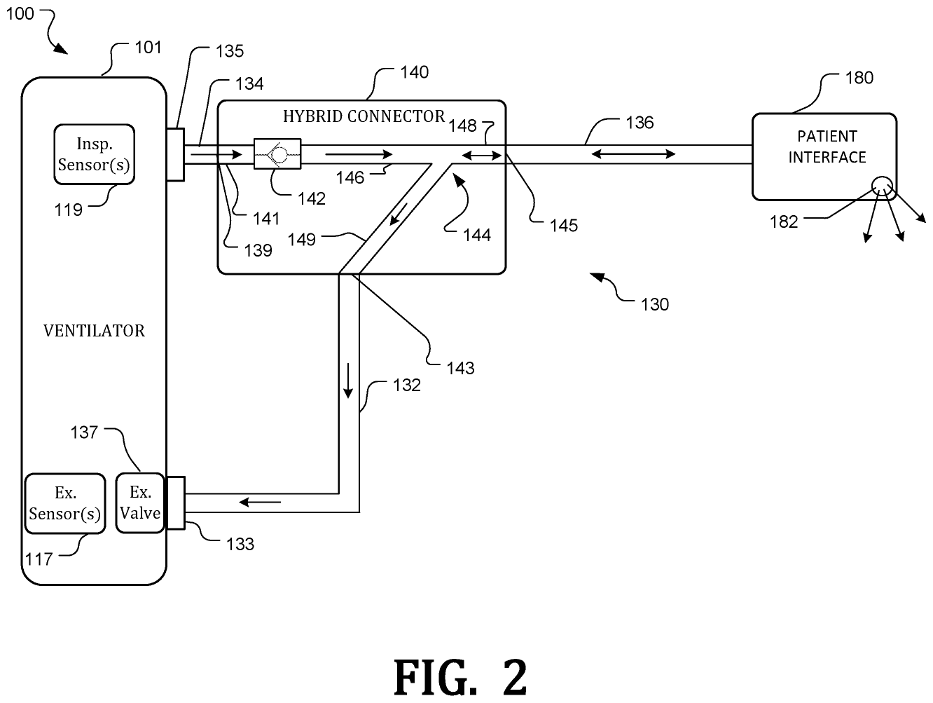

FIG. 2 depicts a schematic diagram of an example medical ventilation system 100 with a hybrid single-limb patient circuit 130 connected to a medical ventilator 101. Similar to the configuration of the hybrid single-limb patient circuit 130 depicted in FIG. 1, the hybrid single-limb patient circuit 130 in FIG. 2 includes an inhalation tubing segment 134 coupled to the inspiratory port 135, an exhalation tubing segment 132 coupled to the expiratory port 133, and a dual-purpose single limb 136. The inhalation tubing segment 134 couples to a ventilator end (e.g., end closer to the ventilator 101) of a hybrid adapter 140, and the dual-purpose single limb 136 couples to the distal end of the hybrid adapter 140. The exhalation tubing segment 132 couples to a side or other end of the hybrid adapter 140. In other examples, the inhalation tubing segment 134, dual-purpose single limb 136, and exhalation tubing segment 132 may couple to the hybrid adapter 140 in different manners.

The example hybrid adapter 140 depicted in FIG. 2 include a breathing gas input port 139 for receiving breathing gases. The hybrid adapter 140 also includes a check valve 142 that is connected to a pre-valve inhalation segment 141 and a post-valve inhalation segment 146. The pre-valve inhalation segment 141 couples to the inhalation tubing segment 134 to receive breathing gases from the inhalation tubing segment 134. For instance, the hybrid adapter 140 may include a cylindrical connector for connected to the tubing of the inhalation tubing segment 134. Other cylindrical connectors may also be incorporated at the other input and output ports of the hybrid adapter 140 to connect or couple to the respective tubing segments. For instance, the tubing may be couplable or removable from the hybrid adapter 140. The hybrid adapter 140 may also be manufactured to be disposable in some examples.

The check valve 142 operates to allow the flow gas only in a single direction (e.g., towards the patient) such that gas flows through the pre-valve inhalation segment 141 to the post-valve inhalation segment 146, but gas cannot flow from the post-valve inhalation segment 146 to the pre-valve inhalation segment 141. Thus, the check valve 142 prevents exhaled gases from flowing back into the ventilator 101 or any other elements that are connected between the ventilator 101 and the hybrid adapter 140. For instance, the hybrid adapter 140 may be connected to an output of a humidifier 138. By connecting the hybrid adapter 140 to the output of the humidifier 138, the humidifier 138 is also protected from exhaled gases.

The manifold 144 operates to allow fresh breathing gases to flow from the post-valve inhalation segment 146 to a post-manifold single-limb segment 148 during inhalation phases. The post-manifold single-limb segment 148 is coupled to the dual-purpose single limb 136, via a single-limb port 145, to allow for gas flow between the post-manifold single-limb segment 148 and the dual-purpose single limb 136. The manifold 144 also operates to direct exhaled breathing gases from the post-manifold single-limb segment 148 into the adapter exhalation segment 149. The adapter exhalation segment 149 is coupled to the exhalation tubing segment 132, via output port 143, such that exhaled gases flowing through the adapter exhalation segment 149 flow into the exhalation tubing segment 132 and ultimately into the expiratory port 133 of the ventilator 101.

Each of the pre-valve inhalation segment 141, the post-valve inhalation segment 146, the post-manifold single-limb segment 148, and/or the adapter exhalation segment 149 may include or define lumens through which the respective gases flow. The walls of the lumens may be defined by flexible tubes and/or rigid structures that form the body of the hybrid adapter 140. For instance, the lumens may be formed by bores within a housing of the hybrid adapter 140.

During ventilation, in addition to compressors or other inspiratory valves, the ventilator 101 may operate an exhalation valve 137 to control the pressure of the gases within the hybrid single-limb patient circuit 130. The properties of the gases may also be measured through the use of inspiratory sensors 119 and expiratory sensors 117. The inspiratory sensors 119 measure properties of the gases flowing through the inspiratory port 135, and the gas property sensors 118 measure properties of the gases flowing through the expiratory port 133. Additional gas property sensors may also be incorporated at other positions within the hybrid single-limb patient circuit 130. For example, one or more gas property sensors may be incorporated into the hybrid adapter 140. As another example, one or more gas property sensors may be incorporated into the dual-purpose single limb 136.

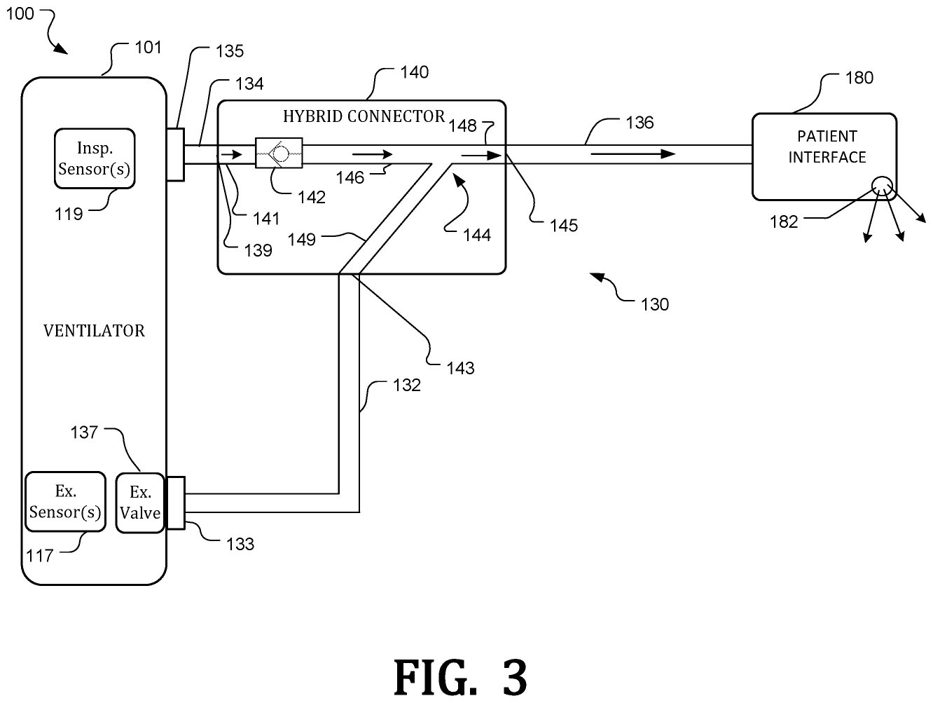

FIG. 3 depicts a schematic diagram of the medical ventilation system 100 of FIG. 2 during an inhalation phase of a breath. During an inhalation phase of a breath, the ventilator 101 delivers breathing gas to the patient interface 180. More specifically, in the example depicted, breathing gases are delivered through the inspiratory port 135 and the inhalation tubing segment 134 into the hybrid adapter 140. Within the hybrid adapter 140, the breathing gases travel through the pre-valve inhalation segment 141, the check valve 142, and the post-valve inhalation segment 146. The breathing gases then flow through the manifold 144 into the post-manifold single-limb segment 148, where the breathing gases exit the hybrid adapter 140 into the dual-purpose single limb 136. During inhalation phases, the exhalation valve 137 is closed or substantially closed, which prevents the breathing gases from flowing into the adapter exhalation segment 149 and the exhalation tubing segment 132.

Once the breathing gases exit the hybrid adapter 140, the breathing gases are carried to the patient interface 180, where the patient breathes the breathing gases. While the breathing gases are delivered to the patient during the inhalation phase, some of the breathing gases may be leaked (e.g., not be inhaled by the patient). Leaks may exist at any point in the hybrid single-limb patient circuit 130 and/or may be the result of gases escaping through a vent port 182 of the patient interface 180.

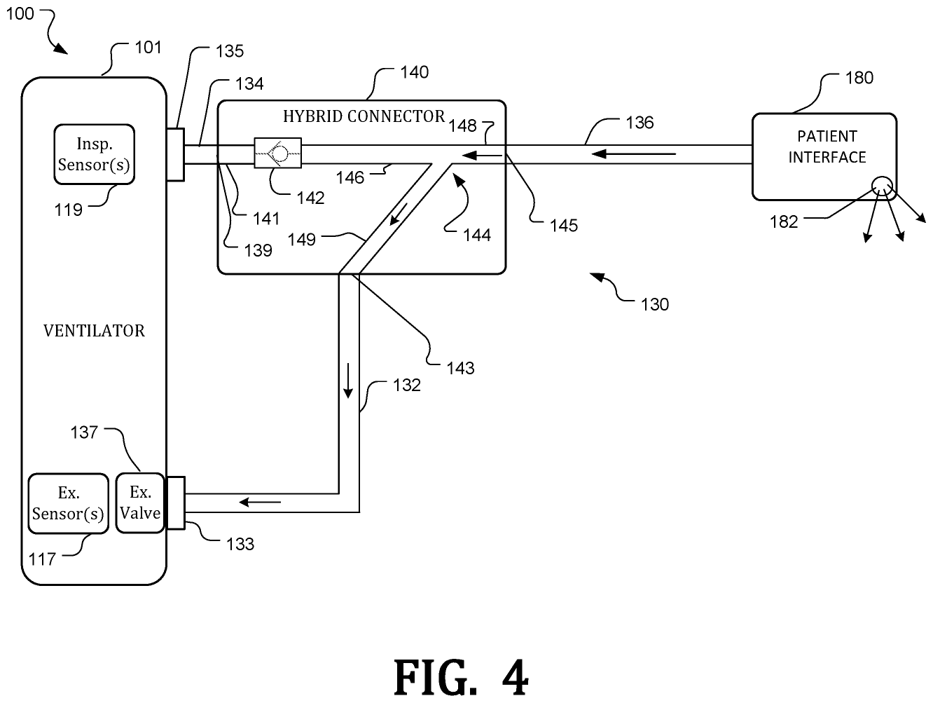

FIG. 4 depicts a schematic diagram of the medical ventilation system 100 of FIG. 2 during an exhalation phase of a breath. During an exhalation phase of a breath, the patient exhales breathing gases into the patient interface 180. A portion of the breathing gases may be vented from the patient interface 180 via the vent port 182. The remaining portion of the exhaled gases may be carried by the dual-purpose single limb 136 back to the hybrid adapter 140. Within the hybrid adapter 140, the exhaled gases are carried by the post-manifold single-limb segment 148 towards the manifold 144, where the exhaled gases are directed through the adapter exhalation segment 149. Due to the positioning of the check valve 142, the exhaled gases are prevented from flowing through the check valve 142 and back into the pre-valve inhalation segment 141.

In addition, during the exhalation phase, the exhalation valve 137 opens to allow for gas flow through the expiratory port 133. Thus, the exhaled gases flow through the adapter exhalation segment 149 and enter the exhalation tubing segment 132. The exhaled gases are then carried by the exhalation tubing segment 132 into the expiratory port 133, where properties of the exhaled gases may be sensed or measured by the expiratory sensors 117. For instance, a flow and/or pressure of the exhaled gases may be measured. The inspiratory sensors 119 and the expiratory sensors 117 may operate during one or both of the inhalation phases and exhalation phases to measure gas properties of the gases flowing within the hybrid single-limb patient circuit 130.

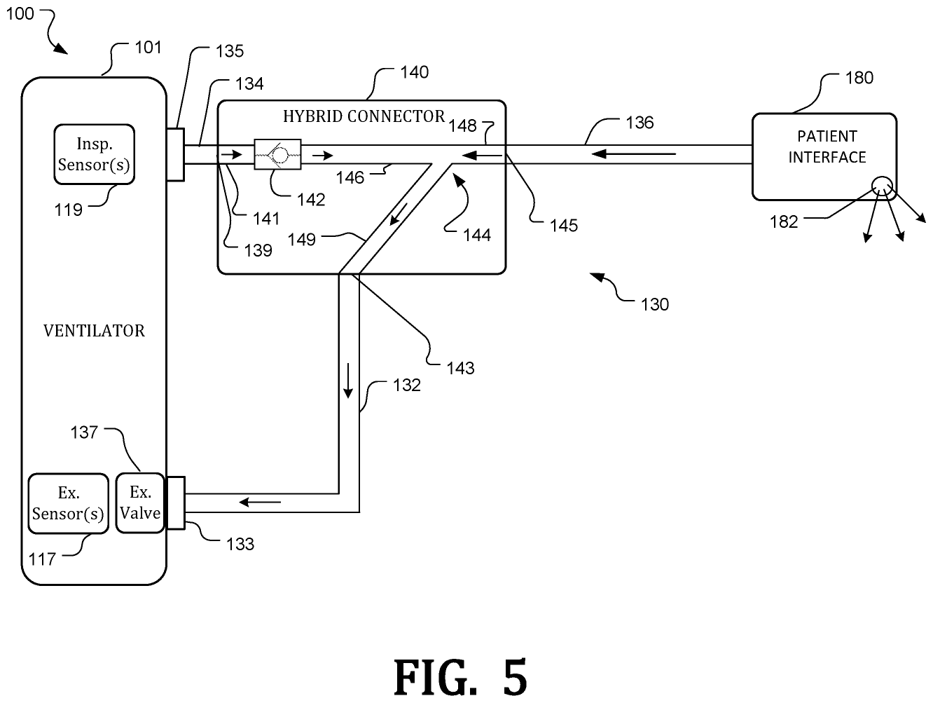

FIG. 5 depicts a schematic diagram of the medical ventilation system 100 of FIG. 2 during an exhalation phase of a breath where positive end expiratory pressure (PEEP) is maintained. As discussed above, during inhalation and exhalation phases, the flow of the gases may be measured using the inspiratory sensors 119 and the expiratory sensors 117. For example, an inspiratory flow sensor measures the flow of the breathing gases delivered through the inspiratory port 135, and an expiratory flow sensor measures the flow of the exhaled gases received in the expiratory port 133.

Based on the flow measurements, an amount of leak occurring in the hybrid single-limb patient circuit 130 may be determined, such as the leak due to the vent port 182 in the patient interface 180. As an example, a volume of gas that is supplied may be determined based on measurements of the inspiratory flow sensor (e.g., integrating flow over the inhalation phase). During exhalation, a volume of exhaled gases (e.g., exhaled tidal volume (VTE)) may also be determined. Based on the volume of gas that is supplied during inhalation and the volume of the exhaled gases, an amount of leak may be determined due to the difference in the volumes. With the leak rate identified, a more accurate estimate of the actual inspired and expired breathing volumes (e.g., inspired tidal volume (VTI), exhaled tidal volume (VTE)) may be determined.

In addition, with the determined leak rate, a PEEP level may be maintained during exhalation phases with the hybrid single-limb patient circuit 130. Generally, maintaining PEEP involves control of the exhalation valve 137 during exhalation to maintain a pressure within the hybrid single-limb patient circuit 130 by controlling flow rate of exhaled gases through the expiratory port 133. Where there is a leak, particularly a large leak such as due to the vent port 182, additional gas is added to the hybrid single-limb patient circuit 130 to maintain the pressure of the hybrid single-limb patient circuit 130 at or above a set PEEP level.

Accordingly, pressure measurements may be made, such as by a pressure sensor of the expiratory sensors 117, to determine a pressure in the hybrid single-limb patient circuit 130 during exhalation. To maintain the pressure above the PEEP level, the position of the exhalation valve 137 is adjusted and an amount of breathing gases supplied through the inspiratory port 135 may also be adjusted based on the determined leak rate. Even though the check valve 142 is technically open while the breathing gases are being provided to maintain PEEP, the components before the hybrid adapter 140 are still protected from contamination because there is positive flow towards the check valve 142 due to by the active flow controller and the exhalation valve 137 is open to drop the circuit pressure towards PEEP. That is, no exhaled gases flow backwards through the check valve 142.

Additionally or alternatively, an amount of exhaled gas that is rebreathed by the patient may also be estimated based on the volume of gases supplied during inhalation and/or the volume of gases returned through the expiratory port 133 and/or vented through the interface vent port 182 during exhalation. The amount of rebreathed gas may be indicative of an amount of carbon dioxide that the patient inhales due to the exhaled gas being rich in carbon dioxide. The amount of gas that may be rebreathed is based on the volume and compliance of the hybrid single-limb patient circuit 130, and particularly the volume of the dual-purpose single limb 136. For instance, the volume of gases within the hybrid single-limb patient circuit 130 shifts during inhalation and exhalation. During a full inhalation or exhalation, the total volume of gas in the hybrid single-limb patient circuit 130 may be cleared (potentially multiple times). During a transition from exhalation to inhalation, however, a volume of exhaled gas may remain present in the dual-purpose single limb 136 when inhalation is initiated. A portion of this exhaled gas may then be inspired by the patient. Through the flow measurements discussed herein and an estimate of the compliance and/or volume of the hybrid single-limb patient circuit 130 (or dual-purpose single limb 136), an estimate of volume of the exhaled gas that is rebreathed by the patient may be generated.

Accordingly, as shown in FIG. 5, a flow of breathing gases is provided through the inspiratory port 135 even during the exhalation phase to maintain PEEP and compensate for gases lost due to the leak. The flow rate of the breathing gases delivered during the exhalation phase is less than the flow rate of breathing gases delivered during the inhalation phase, which allows the patient to exhale without significantly increasing the work of breathing for the patient.

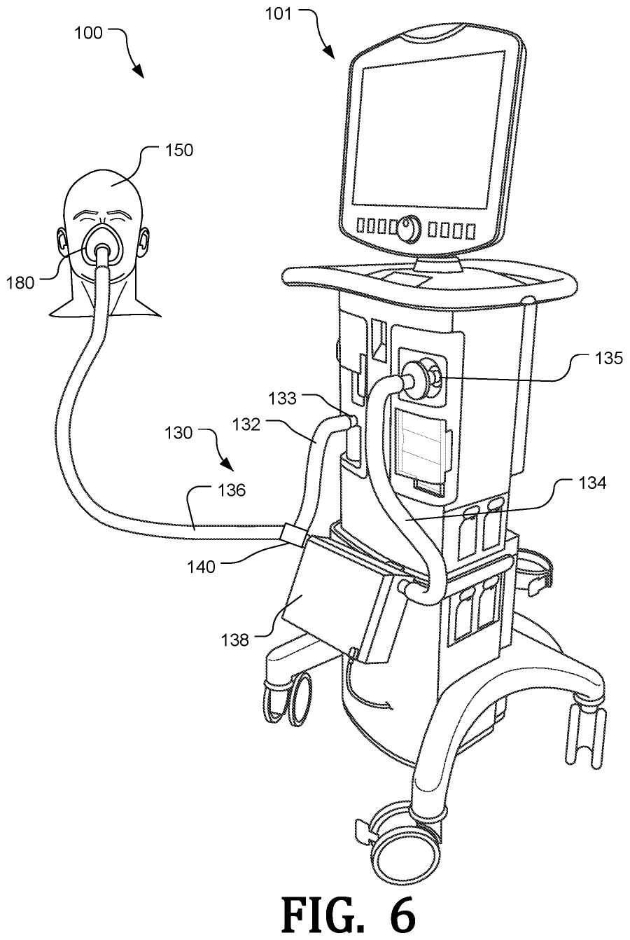

FIG. 6 depicts a perspective view of a medical ventilation system 100 connected to a human patient 150 using a hybrid single-limb patient circuit 130. The system is substantially similar to other systems discussed herein. For instance, the medical ventilation system 100 includes a ventilator 101 that has an inspiratory port 135. An inhalation tubing segment 134 is coupled to the inspiratory port 135 to carry breathing gases from the inspiratory port 135 to a humidifier 138. The inhalation tubing segment 134 couples to an input port of the humidifier 138.

The humidifier 138 humidifies the breathing gases received at the input of the humidifier 138, and outputs humidified gas at the output of the humidifier 138. In the example depicted, the hybrid adapter 140 is coupled to the output of the humidifier 138. As discussed above, a check valve of the hybrid adapter 140 allows for gas flow in only a single direction. Thus, gases cannot flow back into the humidifier 138. In examples, the hybrid adapter 140 may be configured to be directly coupled to an output of the humidifier 138.

A dual-purpose single limb 136 is coupled to the hybrid adapter 140 and extends to the patient interface 180 that is worn by the patient 150. The dual-purpose single limb 136 carries the humidified breathing gases from the hybrid adapter 140 to the patient interface 180. The dual-purpose single limb 136 also carries exhaled gases back to the hybrid adapter 140. Due to the manifold and the check valve in the hybrid adapter 140, the exhaled gases are diverted into the exhalation tubing segment 132, which carries the exhaled gases into the expiratory port 133.

In some examples, the dual-purpose single limb 136 is significantly longer than the exhalation tubing segment 132 and/or the inhalation tubing segment 134. For instance, the dual-purpose single limb 136 may be at least two times as long as the exhalation tubing segment 132 and/or the inhalation tubing segment 134.

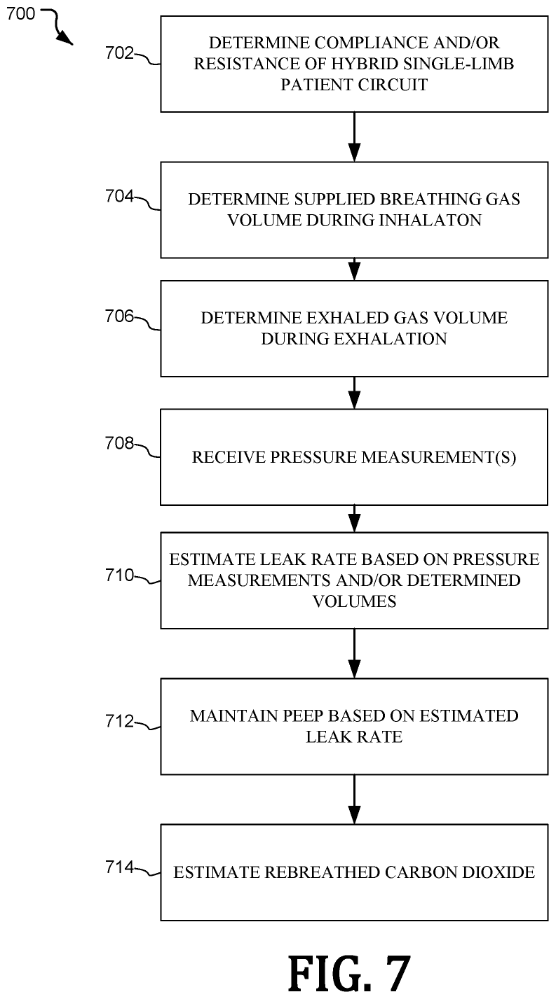

FIG. 7 depicts an example method 700 for delivering ventilation with a hybrid single-limb patient circuit. The example method 700 maybe performed by a ventilator and/or components thereof, such as a processor or controller of the ventilator.

At operation 702, a compliance and/or resistance of the hybrid single-limb patient circuit may be determined. The compliance and/or resistance may be determined from a ventilator self-test that may be performed upon connection of the hybrid single-limb patient circuit to the ventilator. The compliance and/or resistance may be determined for segments of the circuit, such as an inspiratory portion of the circuit and/or an expiratory portion of the patient circuit.

At operation 704, a volume of supplied breathing gas through the inspiratory port is determined. The volume of supplied breathing gas may be determined based on flow measurements at the inspiratory port. The flow through the inspiratory port may also be known or set by the ventilator. In such examples, the volume and or flow rate may be provided by the ventilator rather than having to be measured by a flow sensor at the inspiratory port.

At operation 706, a volume of exhaled gas that is received through the expiratory port is determined. The volume of exhaled gas may be determined based on flow measurements from a flow sensor at the expiratory port. For instance, the expiratory flow sensor measurements may be integrated over the exhalation phase to determine an exhaled gas volume.

At operation 708, one or more pressure measurements may be received. For instance, a pressure measurement from the inspiratory pressure sensor and/or a pressure measurement from an expiratory sensor may be received. The circuit pressure in the hybrid single-limb patient circuit may then be estimated from the inspiratory pressure measurement and the expiratory pressure measurement. The circuit pressure may be an estimate of the pressure at the patient interface (e.g., at a point where the patient interface connects to the dual-purpose single limb).

Determining the circuit pressure at the patient interface may be performed in a variety of manners. As an example, the circuit pressure may be determined based on an inspiratory pressure sensor measurement, the supplied gas flow rate (e.g., a flow measurement from the inspiratory flow sensor), and a resistance of the inspiratory portion of the circuit (determined in operation 702), which may include the inhalation tubing segment, the hybrid connector, the dual-purpose single limb, the humidifier (where included), and any other components on the inspiratory pathway. The circuit pressure at the patient interface is then equal to the inspiratory pressure measurement minus the pressure drop through the inspiratory side of the circuit, which is based on the inspiratory flow rate and the resistance of the inspiratory side (e.g., inspiratory flow multiplied by inspiratory resistance). As another example, the circuit pressure may be determined based on an expiratory sensor measurement, the exhaled flow rate (e.g., a flow measurement from the expiratory flow sensor), and a resistance of the expiratory portion of the circuit (determined in operation 702), which may include the exhalation tubing segment, the hybrid connector, the dual-purpose single limb, and any other components on the expiratory pathway. The circuit pressure at the patient interface is then equal to the expiratory pressure measurement plus the pressure drop across expiratory side of the circuit, which is based on the expiratory flow rate and the resistance of the expiratory side (e.g., expiratory flow multiplied by expiratory resistance).

At operation 710, a leak rate may be estimated based on the exhaled volume determined in operation 706, the supplied volume determined in operation 704, and/or the estimated circuit pressure at the patient interface determined in operation 708. In some examples, the difference between the supplied volume and exhaled volume may be used to determine one or more constants that characterize a leak in the circuit and/or at the patient interface (such as through the vent port). The circuit pressure may then be used to determine a leak rate at any particular point in time. For instance, when the circuit pressure is higher, the leak rate may be higher, and when the circuit pressure is lower, the leak rate may be lower. Accordingly, a current leak rate may be estimated based on a current circuit pressure. With the leak rate known or estimated, an estimate of the inspired tidal volume (e.g., an amount of breathing gases actually inhaled by the patient) may be generated. For example, the inspired tidal volume will be less than the supplied volume of breathing gases due to the presence of the leak.

At operation 712, a PEEP level is maintained during an exhalation phase based on the leak rate estimated in operation 710. For example, the amount of closure of the expiratory valve is adjusted during exhalation to maintain a set PEEP level. Due to the leak rate, however, additional gas may be required to maintain the set PEEP level. Accordingly, based on the leak rate, additional gas is supplied through the inspiratory port to maintain the PEEP level. The flow rate of the supplied gases to maintain PEEP is less than that of the flow rate of the supplied gases during inhalation.

At operation 714, an estimate of rebreathed carbon dioxide may be made based on the compliance determined at operation 702 as well the supplied breathing gas volume determined in operation 704, the exhaled gas volume determined in operation 706, and/or the estimated leak in operation 710. For instance, an amount of exhaled gases that are rebreathed through the dual-purpose single limb may be estimated. Based on an estimated or measured carbon dioxide concentration of the breathing gases, the amount of carbon dioxide that has been rebreathed may then be estimated.

As an example, based on the compliance of the circuit (determined in operation 702) and a circuit pressure, the volume of gas in the circuit may be determined. Based on the inspiratory flow measurements over time, the volume of gas supplied may be determined, and based on the expiratory flow measurements, the volume of gas received through the exhalation port may be estimated. The volume of gas that is vented through the vent port or other leak source may also be estimated during the inspiratory phase and the expiratory phase. For instance, during an exhalation phase, an estimate of the volume of gas that is vented and the volume of gas that is received through the exhalation port is received. A volume of exhaled gas remaining in the dual-purpose single limb may then be estimated based on those volumes and/or the circuit pressure and the compliance of the circuit. When the inspiratory phase is initiated, that volume of exhaled gas is delivered back to the patient interface, where a portion of the exhaled gas is inhaled and a portion of the exhaled gas is vented through the vent port. The portion of the exhaled gas that is vented may be determined based on the determined leak rate. Thus, the portion of volume of exhaled gas that is rebreathed by the patient may be determined by subtracting the portion of exhaled gases that are vented or leaked.

A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

1. A ventilation system comprising:
a ventilator comprising:
an inspiratory port;
an expiratory port;
an exhalation valve positioned at the expiratory port;
a non-invasive patient interface; and
a hybrid single-limb patient circuit coupled to the inspiratory port and the expiratory port, the hybrid single-limb patient circuit comprising:
a check valve positioned to direct breathing gases supplied from the inspiratory port in a single direction;
a manifold pneumatically coupled to the check valve;
a dual-purpose single limb, pneumatically coupled to the manifold and the non-invasive patient interface, to carry breathing gases to the non-invasive patient interface and carry exhaled gases from the non-invasive patient interface; and
an exhalation tubing segment, pneumatically coupled to the manifold and the expiratory port, to carry the exhaled gases from the manifold to the expiratory port.

2. The ventilation system of claim 1, wherein the manifold comprises:
a post-valve inhalation segment to carry the breathing gases passing through the check valve;
a post-manifold single-limb segment to carry the breathing gases from the post-valve inhalation segment to a single-limb port and carry the exhaled gases received from the single-limb port; and
an adapter exhalation segment to carry the exhaled gases to the exhalation tubing segment.

3. The ventilation system of claim 1, further comprising a humidifier, wherein the check valve is coupled to an output of the humidifier.

4. The ventilation system of claim 1, wherein the ventilator further comprises:
an expiratory flow sensor;
an expiratory pressure sensor;
an inspiratory pressure sensor;
an inspiratory flow sensor;
a processor; and
memory, the memory storing instructions that, when executed by the processor, cause the ventilator to perform operations comprising:
based on measurements from the expiratory flow sensor, estimate an exhaled volume of gas;
estimate a volume of supplied breathing gas; and
based on the estimated exhaled volume of gas, the estimated volume of supplied breathing gas, and at least one of an inspiratory pressure measurement from the inspiratory pressure sensor or an expiratory pressure measurement from the expiratory sensor, estimating a leak rate.

5. The ventilation system of claim 4, wherein the operations further comprise:
maintain, during an exhalation phase, a positive end expiratory pressure (PEEP) level by:
controlling an exhalation valve; and
supplying breathing gases through the inspiratory port at a flow rate based on the estimated leak rate.

6. The ventilation system of claim 4, wherein the operations further comprise:
estimate a compliance of the hybrid single-limb patient circuit; and
based on the compliance, an expiratory flow measurement from the expiratory sensor, the exhaled volume of gas, and the leak rate, estimating a volume of rebreathed carbon dioxide.

7. A hybrid adapter for facilitating hybrid single-limb ventilation, the hybrid adapter comprising:
a breathing gas input port to receive breathing gases from a ventilator;
a pre-valve inhalation segment coupled to the breathing gas input port;
a check valve, coupled to the pre-valve inhalation segment, to direct the breathing gases in a single direction;
a manifold;
a single-limb port, coupled to the manifold, to deliver the breathing gases to a couplable dual-purpose single limb and receive exhaled breathing gases from the couplable dual-purpose single limb; and
an output port, coupled to the manifold, to deliver the exhaled gases to a couplable exhalation tubing segment.

8. The hybrid adapter of claim 7, wherein the manifold comprises:
a post-valve inhalation segment coupled to the check valve;
a post-manifold single-limb segment extending from the post-valve inhalation segment to the single-limb port; and
an adapter exhalation segment extending from the post-manifold single-limb segment to the output port.

9. The hybrid adapter of claim 8, wherein the pre-valve inhalation segment, the post-valve inhalation segment, the post-manifold single-limb segment, and the adapter exhalation segment comprise tubing.

10. The hybrid adapter of claim 8, wherein the pre-valve inhalation segment, the post-valve inhalation segment, the post-manifold single-limb segment, and the adapter exhalation segment comprise bores in a housing of the hybrid adapter.

11. A ventilator-implemented method, the method comprising:
estimating a volume of supplied breathing gas flowing from an inspiratory port of a ventilator and into a hybrid single-limb patient circuit, the hybrid single-limb patient circuit comprising a dual-purpose single limb defining a lumen that carries both delivered breathing gases and returned exhaled gases;
estimating a volume of exhaled gas flowing from the hybrid single-limb patient circuit into an expiratory port of the ventilator;
estimating a circuit pressure of the hybrid single-limb patient circuit at a patient interface;
based on the estimated exhaled volume of gas, the estimated volume of supplied breathing gas, and the circuit pressure, estimating a leak rate; and
based on the estimated leak rate, maintaining a positive end expiratory pressure (PEEP) level in the hybrid single-limb patient circuit during an exhalation phase.

12. The ventilator-implemented method of claim 11, wherein estimating the volume of exhaled gases is based on measurements from an expiratory flow sensors positioned at the expiratory port.

13. The ventilator-implemented method of claim 11, wherein maintaining PEEP comprises:
controlling a closure rate of an exhalation valve at the expiratory port; and
supplying breathing gases through the inspiratory port at a flow rate based on the estimated leak rate.

14. The ventilator-implemented method of claim 11, further comprising estimating an amount of rebreathed carbon dioxide based on the leak rate and at least one of volume of supplied breathing gas or the volume of exhaled gases.

15. The ventilator-implemented method of claim 14, further comprising determining a compliance of the hybrid single-limb patient circuit, and wherein estimating the amount of rebreathed carbon dioxide is further based on the determined compliance.
